(19) United States
(12) Patent Application Publication
(10) Pub. No.: US 2017/0245819 A1
(43) Pub. Date: Aug. 31, 2017

(54) SYSTEMS AND METHODS FOR IN-VIVO DETECTION OF LEAD IN BONE

(71) Applicants: Peter J. Rothschild, Newton, MA (US); Lee Grodzins, Lexington, MA (US)

(72) Inventors: Peter J. Rothschild, Newton, MA (US); Lee Grodzins, Lexington, MA (US)

(21) Appl. No.: 15/444,135

(22) Filed: Feb. 27, 2017

Related U.S. Application Data
(60) Provisional application No. 62/300,210, filed on Feb. 26, 2016.

(51) Int. Cl. A61B 6/00; A61B 6/06

(52) U.S. Cl. CPC A61B 6/505; A61B 6/4071; A61B 6/4078; A61B 6/06; A61B 6/4241

(57) ABSTRACT

A system and corresponding method for detecting one or more high-atomic-number elements in a patient includes a Bremsstrahlung x-ray source that produces x-rays in an energy spectrum including an energy of at least 160 kiloelectron-volts (keV), a filter configured to absorb the x-rays in a region of the energy spectrum, and a collimator configured to receive the x-rays and output a collimated x-ray beam to be incident on a patient. The system and method can also include one or more collimated, energy-resolving x-ray detectors to detect fluorescent radiation emitted from the one or more high-atomic-number elements in the patient in response to the collimated x-ray beam incident on the patient. An alternative x-ray source can include a radioactive isotope. Scanning of the x-ray beam may also be performed. Embodiments enable practical clinical, in vivo measurements of lead in bone.

### RELATED APPLICATION

This application claims the benefit of U.S. Provisional Application No. 62/300,210, filed on Feb. 26, 2016. The entire teachings of the above application(s) are incorporated herein by reference.

### BACKGROUND

Lead (Pb), which has been known to be a poison since antiquity, is now known to be especially harmful during childhood years, even at concentrations below 10 ppm. Most developed countries of the world outlawed the use of lead in gasoline and in house paint decades ago, but heritage lead remains in the soil and in the paint in older homes. Lead poisoning continues to be a serious health problem as evidenced by media coverage in developing countries such as China, and in parts of the United States.

Currently, lead poisoning is determined by measuring its concentration in blood. These clinical tests are sensitive, quick, and inexpensive. But the concentration in the blood from a poisoning episode drops by about a factor of two each month as the blood lead gets absorbed by the body's bones. A single blood test reveals no conclusive information about the long-term lead stored in the bone, or from a poison episode only a few months old. The true level of long-term exposure can only be assessed by a direct measurement of the amount of lead in the patient's bone. Present in-vivo x-ray fluorescence (XRF) studies of Pb in bones use the radioactive source, Cd¹⁰⁹, which has a half-life of 15.4 months with the emission of an 88.034 keV gamma ray in 4% of the decays. The gamma ray energy is only 29 eV above the 88.005 keV binding energy of the K electron in Pb. (Hereinafter, the abbreviation XRF-PbB is used as a shorthand for in-vivo lead-in-bone XRF for a system that uses a radioactive isotope source, and the abbreviation XRF-XPbB is used for a system that uses an x-ray tube (an example Bremsstrahlung x-ray source) as the source.

Strong radioactive sources of Cd¹⁰⁹, together with high-resolution detectors, have been used for decades for research studies. These XRF-PbB instruments have improved substantially in recent years by using four high-resolution detectors to increase the total count rate so as to reduce the statistical uncertainty and improve the sensitivity. Recent studies measure bone burdens at the 5 ppm level.

FIG. 1 is a photograph of one of the systems measuring the lead burden in a young woman's tibia. A beam of 88 keV γ-rays from a Cd¹⁰⁹ source impinges on about one square cm of the patient's tibia, one of the body's bones with the thinnest overlying tissue. Four un-collimated germanium detectors, positioned around the source, measure the backscattered Compton-scattered x-rays and characteristic K x-rays of lead.

FIG. 2 shows the fluoresced spectrum taken of a bone phantom containing 112 ppm of lead. The signature K x-rays of Pb at 72.6 keV and 75 keV are clearly evident in this 30 minute test using a 100 mCi source of Cd¹⁰⁹. In contrast of FIG. 2, at the 5 ppm level, not shown, the Pb signal shrinks by a factor of 22, and the signature lines will no longer be discernable above the background. An accurate value of the Pb burden requires a sophisticated curve-fitting program of the Compton background that varies with the patient's size and the thickness of tissue that overlays the bone. Further advances to the methodology described in connection with FIGS. 2-3 will doubtless be made. However, techniques that use a radioactive source and take more than a few minutes for a sensitive test will remain a research tool and are not practical for clinical use.

### SUMMARY

A new K-shell x-ray fluorescence (XRF) method is described herein for in-vivo measurements of the concentration of lead (Pb) in a person's bones. The present disclosure involves using, for the first time, an x-ray tube to generate the collimated Bremsstrahlung beam that, in-vivo, fluoresces bones, such as the tibia or patella, which have relatively thin overlying tissue. Precise and extensive simulations show that one can attain the desired sensitivity of 5 ppm of Pb in a test of a few minutes by using an x-ray tube of at least 180 keV, with its spectral shape altered by filters that absorb the Bremsstrahlung spectrum in the range of 90 keV to 130 keV. To reduce the radiation burden to the patient, a further feature of embodiments includes means to inspect an order of magnitude more bone area than has been done previously. One means to do this scans the filtered Bremsstrahlung beam over a length of the tibia. A second means collimates the filtered Bremsstrahlung beam so as to strike a large area of the selected bone and has a detector array that efficiently collects from the large area. Precise simulations show that the combination of these novel features will make practical clinical, in vivo measurements of long-term stores of lead in the patient's bone.

Computer simulations demonstrating the effectiveness of embodiments were mainly run using Geant3.21 rather than Geant4. The accuracy of the Geant3.21 simulations for processes involving gamma rays and x-rays greater than 10 keV has been confirmed over many years and has been found to run substantially faster than Geant4, which includes x-ray energies below 10 keV, which have less relevance to embodiments described herein.

These simulations show that the desired sensitivity of 5 ppm of Pb can be attained in a measurement of a few minutes by using an x-ray tube of preferably at least 180 keV, with its spectral shape altered by filters that strongly absorb the Bremsstrahlung spectrum in the range of 90 keV to 130 keV. To reduce the radiation burden to the patient, further means are described herein to inspect a much larger bone area than previous measurements. One means scans a pencil beam of the filtered Bremsstrahlung beam over a long length of the tibia, or the surface area of the patella. Another means collimates the filtered Bremsstrahlung beam into a fan beam that illuminates a larger area of the selected bone. The combination of these novel inventions will make practical clinical, in vivo, measurements of long-term stores of lead. The specifics chosen and described herein to validate these approaches are exemplar. Those familiar with the art of x-ray imaging will modify parameters, in view of the description herein, according to their needs for various applications.

In one embodiment, a system for detecting one or more high-atomic-number elements in a patient includes a Bremsstrahlung x-ray source configured to produce x-rays in an energy spectrum including an energy of at least 160 keV. The system also includes a filter configured to absorb the x-rays from the x-ray source in a region of the energy spectrum and a collimator configured to receive the x-rays from the x-ray source and to output a collimated x-ray beam to be incident on a patient. The system further includes one or more collimated, energy resolving x-ray detectors configured to detect fluorescent radiation emitted from one or more high-atomic-number elements in the patient in response to the collimated x-ray beam incident on the patient.

The patient can be a human or animal. The fluorescent radiation can be emitted from the high-atomic-number elements in a bone, such as a tibia bone. The system can further include a scanner configured to cause relative motion between the patient and the x-ray beam incident on the patient in order to scan at least a portion of the patient with the x-ray beam. The scanner can be further configured to move the patient with respect to the x-ray beam to cause the relative motion, or to cause relative one-dimensional motion between the patient and the x-ray beam to scan the portion of the patient along one dimension. The scanner can be further configured to cause relative two-dimensional motion between the patient and the x-ray beam to scan the portion of the patient along two dimensions.

The system can further include an analyzer configured to receive signals from the one or more detectors, the signals being representative of the fluorescent radiation emitted and detected. The analyzer can be configured to process the signals to determine a content of the one or more high-atomic-number elements in the patient, such as a concentration. The analyzer can be configured to determine the content of the one or more high-atomic-number elements with concentration of the one or more elements as low as 5 parts per million (ppm).

The filter can be further configured to absorb x-rays from the x-ray source in a region of the energy spectrum corresponding to x-rays Compton scattered from the patient in response to the collimated x-ray beam incident on the patient, such that a signal-to-background ratio of the fluorescent radiation in comparison with other detected radiation is enhanced. Detected fluorescent radiation may result from K-shell excitations in the one or more high-atomic-number elements in the patient, and the filter can be further configured to absorb the x-rays from the x-ray source in the region of the energy spectrum, wherein the region of the energy spectrum includes a region for maximized excitation cross-section for K-shell excitations. The filter can include a material with an atomic number of at least 50, and the filter can also include a material with an atomic number in a range of about 72-92. The filter can have a thickness of at least 0.5 mm.

The one or more high-atomic-number elements can include lead.

The collimated x-ray beam can be a pencil beam, and the one or more collimated detectors can be arranged to detect the fluorescent radiation emitted only from a path of the pencil beam in the patient. As an alternative, the collimated x-ray beam can be a fan beam, and the one or more collimated detectors can be arranged to detect the fluorescent radiation emitted only from a path of the fan beam in the patient.

In another embodiment, a corresponding method for detecting one or more high-atomic-number elements in a patient can include producing Bremsstrahlung x-rays in an energy spectrum including an energy of at least 160 keV. The method further includes filtering to absorb the x-rays from the x-ray source in a region of the energy spectrum, as well as collimating the x-rays from the x-ray source to produce a collimated x-ray beam to be incident on a patient. The method further includes detecting energy resolve, fluorescent radiation emitted from one or more high-atomic-number elements in the patient in response to the collimated x-ray beam incident on the patient. The fluorescent radiation emitted can be x-ray fluorescent radiation.

The patient can be a human or animal. The fluorescent radiation can be emitted from the high-atomic-number elements in a bone, such as a tibia bone.

The method can further include scanning at least a portion of the patient with the x-ray beam by causing relative motion between the patient and the x-ray beam incident on the patient. Scanning can include moving the patient with respect to the x-ray beam to cause the relative motion, or causing relative one-dimensional motion between the patient and the x-ray beam. The scanning can be two-dimensional and can include causing relative two-dimensional motion between the patient and the x-ray beam.

The method can further include analyzing signals representative of detected, energy resolved, fluorescent radiation emitted from the one or more high-atomic-number elements in the patient to determine a content of the one or more high-atomic-number elements patient, where the content can include concentration. Analyzing can also include determining the content of the one or more high-atomic-number elements with concentration as low as 5 ppm.

Filtering can include absorbing the x-rays from the x-ray source in a region of the energy spectrum corresponding to x-rays Compton scattered from the patient in response to the collimated x-ray beam incident on the patient, such that a signal-to-background ratio can be enhanced. The fluorescent radiation can result from K-shell excitations in the one or more high-atomic-number elements in the patient in response to the x-ray beam incident on the patient, and filtering can include reducing x-rays from the x-ray source in the region of the energy spectrum, where the region is a region of maximum cross section for K-shell excitation by the incident x-rays. Filtering can include using a filter material with an atomic number of at least 50, or a filter with an atomic number in a range of about 72-92. Filtering can include using a filter material having a thickness of at least 0.5 mm. The one or more high-atomic-number elements can include lead.

Collimating the x-rays can include producing a pencil beam or a fan beam. Detecting energy resolved, fluorescent radiation may include detecting the fluorescent radiation emitted only from a path of the pencil beam or fan beam in the patient.

In yet another embodiment, a system for detecting one or more high-atomic-number elements in a patient includes means for producing Bremsstrahlung x-rays in an energy spectrum including an energy of at least 160 keV. The means for producing Bremsstrahlung x-rays may include an x-ray tube, such as a stand-alone x-ray tube or a mono block x-ray tube. The system also includes means for filtering to absorb the x-rays from the x-ray source in a region of the energy spectrum, as well as means for collimating the x-rays from the x-ray source to produce a collimated x-ray beam to be incident on a patient. The system further includes means for detecting energy resolved, fluorescent radiation emitted from one or more high-atomic-number elements in the patient in response to the collimated x-ray beam incident on the patient.

In still a further embodiment, a system for detecting one or more high-atomic-number elements in a patient includes an x-ray source configured to produce x-rays, a collimator configured to receive the x-rays from the x-ray source and to output a collimated x-ray beam to be incident on a patient, and a scanner configured to cause relative motion between the patient and the x-ray beam incident on the patient in order to scan at least a portion of the patient with the x-ray beam. The system also includes one or more collimated, energy resolving x-ray detectors configured to detect fluorescent radiation emitted from one or more high-atomic-number elements in the patient in response to the collimated x-ray beam incident on the patient.

The patient can be a human or animal. The fluorescent radiation can be emitted from the high-atomic-number elements in a bone, such as a tibia bone.

The scanner may be configured to move the patient with respect to the x-ray beam to cause the relative motion, or configured to translate the x-ray beam with respect to the patient. The scanner may be configured to cause relative one-dimensional motion between the patient and the x-ray beam to scan the portion of the patient along one dimension. The scanner may be configured to cause relative two-dimensional motion between the patient and the x-ray beam to scan the portion of the patient along two dimensions. The x-ray source can be a radioactive isotope or an x-ray tube.

The system can also include an analyzer configured to receive signals from the one or more detectors, the signals being representative of the fluorescent radiation emitted and detected, wherein the analyzer is further configured to process the signals to determine a content of the one or more high-atomic-number elements in the patient. The analyzer can be configured to determine the content, such as concentration, of the one or more high-atomic-number elements with concentration as low as 5 ppm. The one or more high-atomic-number elements can include lead.

In yet another embodiment, a method for detecting one or more high-atomic-number elements in a patient includes providing a source of x-rays, collimating the x-rays from the x-ray source to produce a collimated x-ray beam to be incident on a patient, and scanning at least a portion of the patient with the x-ray beam by causing relative motion between the patient and the x-ray beam incident on the patient. The method also includes detecting energy resolved, fluorescent radiation emitted from one or more high-atomic-number elements in the patient in response to the collimated x-ray beam incident on the patient.

The patient can be a human or animal, including a living human or animal. The fluorescent radiation can be emitted from the high-atomic-number elements in a bone, such as a tibia bone.

Scanning may include moving the patient with respect to the x-ray beam, or translating the x-ray beam with respect to the patient, to cause the relative motion. Scanning may be one-dimensional and include causing relative one-dimensional motion between the patient and the x-ray beam, and scanning may be two-dimensional and include causing relative two-dimensional motion between the patient and the x-ray beam.

The x-ray source may be a radioactive isotope or an x-ray tube.

The method can further include analyzing signals representative of detected, energy resolved, fluorescent radiation emitted from the one or more high-atomic-number elements in the patient to determine a content, such as concentration, of the one or more high-atomic-number elements in the patient. Analyzing can also include determining the content of the one or more high-atomic-number elements with concentration as low as 5 ppm. The one or more high-atomic-number elements can include lead.

In still a further embodiment, a system for detecting one or more high-atomic-number elements in a patient includes means for providing a source of x-rays, means for collecting the x-rays from the x-ray source to produce a collimated x-ray beam to be incident on a patient, and means for scanning at least a portion of the patient with the x-ray beam by causing relative motion between the patient and the x-ray beam incident on the patient. The system also includes means for detecting energy resolved, fluorescent radiation emitted from one or more high-atomic-number elements in the patient in response to the collimated x-ray beam incident on the patient.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a photograph of an isotope-source-based prior-art XRF-PbB system intended to measure the lead burden in a young woman's tibia.

FIG. 2 is fluoresced XRF-PbB spectrum taken by the prior-art system of FIG. 1, with a bone phantom containing 112 parts per million (ppm) of lead (Pb) as a sample.

FIG. 3 is simulated spectrum from a 200 kV, tungsten (W) anode X-ray tube, without filtration.

FIGS. 4A-4C are simulated spectra from a 200 kV, W anode X-ray tube filtered by 0.5 mm, 0.7 mm, and 1.0 mm of W, respectively.

FIGS. 5A-5B are simulated energy spectra (from 70 keV to 90 keV) from an XRF-XPbB system, of a bone with 225 ppm of Pb, for a 225 kV (1.0 mm W filter) (FIG. 5A) and a 320 keV (1.7 mm W filter) (FIG. 5B) W anode tube of the same power.

FIG. 6 is an illustration of an embodiment system for determining content of high-atomic-number elements in a patient, wherein the system includes an actuator for translating the x-ray beam with respect to the patient to perform scanning.

FIG. 7 is a drawing illustrating certain components of a pencil beam-based scanning system to increase the scan area and, thus, reduce the radiation burden of an XRF-XPbB system.

FIG. 8 is an illustration of certain components of an XRF-PbB system utilizing a fan beam.

FIG. 9 is an illustration of an embodiment system for determining content of high-atomic-number elements in a patient, wherein the system includes a filter for the x-ray beam.

FIG. 10 is an illustration of an embodiment system for determining content of high-atomic-number elements in a patient, wherein the system can include an radioactive isotope based x-ray source.

FIG. 11 is a flow diagram illustrating an embodiment method for determining content of one or more high-atomic-number elements in a patient, the method includes filtering.

FIG. 12 is a flow diagram illustrating an embodiment method for determining content of one or more high-atomic-number elements in a patient, the method including scanning.

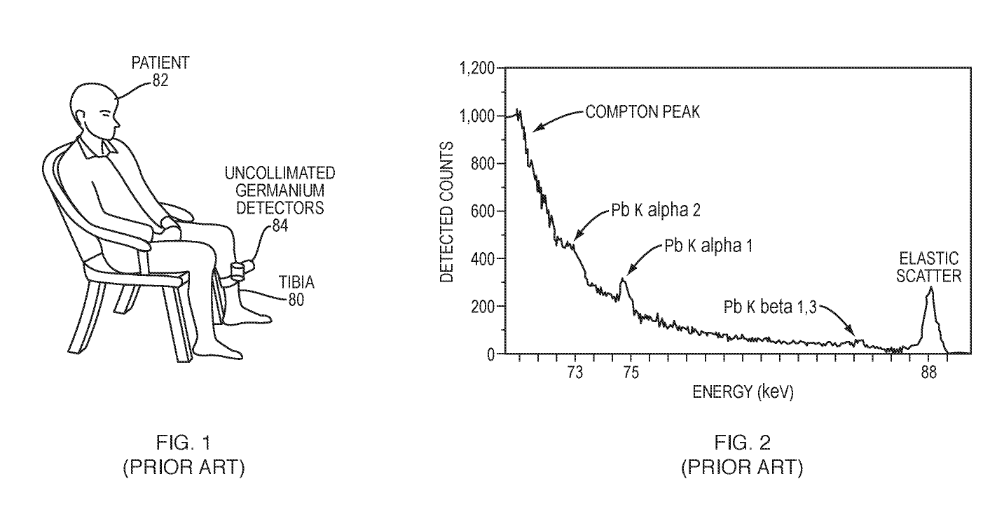

### DETAILED DESCRIPTION

A description of example embodiments of the invention follows.

The intensity of 88 keV gamma rays from a Curie of Cd¹⁰⁹ is a very weak source for stimulating fluorescence from lead when compared to even a small x-ray tube. Compared to the intensity of the 88 keV gamma rays from a one Curie source of Cd¹⁰⁹, each watt of beam power from a 200 keV x-ray tube produces about 100 times more x-rays above 90 keV. At 100 Watts, a compact x-ray tube produces approximately 100 thousand times the intensity of the 0.1 Ci sources now used for XRF-PbB. This basic fact makes practical the use of strong absorbers to shape the spectrum from a commercially available x-ray tube to obtain the necessary sensitivity in a desired short measurement time for clinical use. And it further allows different detector modalities that reduce the radiation dose burden of an in vivo examination.

### Shaping the Bremsstrahlung Spectrum:

The region from 90 keV to 130 keV contains about 14% of the Bremsstrahlung spectrum for an x-ray tube operating at 200 kV. The K-shell photoelectric (PE) cross section for Pb is 10 cm²/g at the K binding energy of 88 keV and diminishes approximately as the cube of the fluorescing energy. At 120 keV, the PE cross section has dropped to 3 cm²/g and at 200 keV it is only 0.8 cm²/g. It is therefore natural to assume that the fluorescing spectrum of the primary beam should be maximized in the 88 keV-130 keV energy region to make use of the highest photo-electric cross section to excite the lead atoms, and to consequently maximize the fluorescence signal coming from the lead. In fact, when the goal is to measure sensitivities in the 5 ppm region, the opposite is true. This is a non-obvious and unexpected result, and the key to understanding this is the following: If x-rays in the energy range of approximately 88 keV-130 keV region in the primary beam undergo just a single Compton scatter event (which has a large probability of occurring), then they will be down-shifted in energy just enough to lie directly under the lead fluorescence peaks in the 72 keV-85 keV region. This creates a large background under the lead peaks, reducing the Signal-to-Noise Ratio (SNR) and reducing the sensitivity of the system.

Suppressing the incident flux in that incident region of approximately 88 keV to 130 keV of the primary beam sharply reduces the total probability for the K-shell excitation of lead, but the loss in signal is more than compensated for by relying on x-rays above 130 keV to excite the lead K-shell x-rays. This is because x-rays in the primary beam above 130 keV must be Compton scattered at least twice before being detected if they are to lie under the lead fluorescence peaks and contribute to the background, interfering with fluorescence to be detected. Since the probability of a double Compton scatter event is much lower than the probability of a single Compton scatter event, the background under the lead signal peaks is much smaller. Even though the lead excitation is also considerably lower, the overall SNR for detecting the lead in low concentrations is greatly improved.

Shaping the input spectrum can be done in a variety of ways. Filters made of Tungsten (W) are: effective, as are the higher atomic number rare earths, e.g. Erbium. Even Pb can be used if precautions are taken to keep its fluorescence K x-rays out of the targeted incident Bremsstrahlung spectrum or out of the detector itself. And combinations of different atomic number can be used to suppress the radiation fluoresced from the filters.

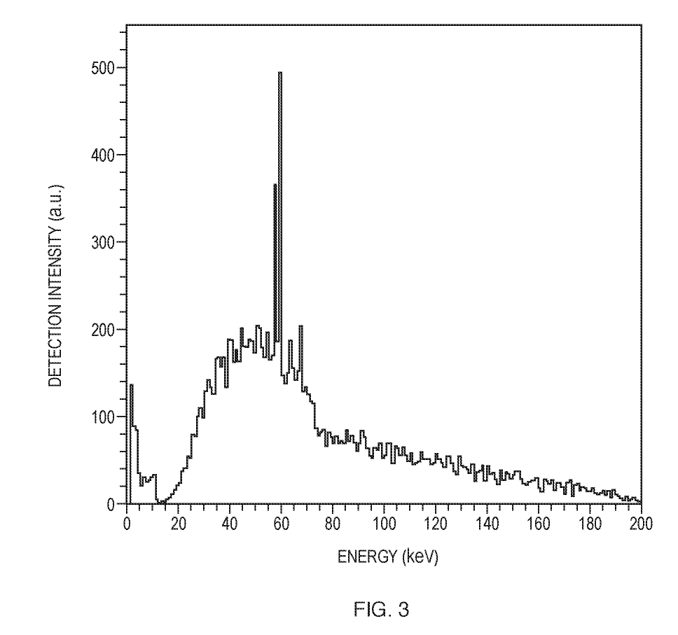

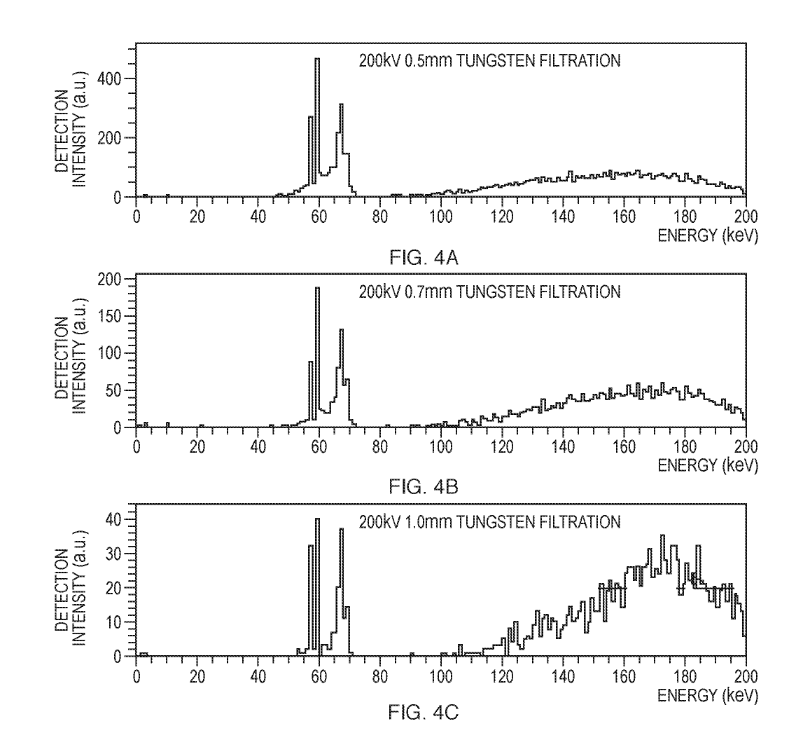

The results of simulations with tungsten absorbers are illustrated in FIGS. 3 and 4, obtained with an x-ray tube operating voltage of 200 kV. FIG. 3 is an illustration of the spectrum with no filtration. The spectra in FIGS. 4A-4B include filtration with filters of thickness 0.5 mm, 0.7 mm, and 1 mm of tungsten, respectively.

The filtration has dramatically shaped the Bremsstrahlung beam (an x-ray beam produced by a Bremsstrahlung radiative process) in FIGS. 4A-4C, relative to the unfiltered spectrum of FIG. 3.

Table 1 shows the percentages in the region from 90 keV to 130 keV compared to the percentages in the region from 130 keV to 200 keV as the tungsten filter increases in thickness. One mm of tungsten has created a Bremsstrahlung beam that is dominated by the high energy region above 130 keV, with a much diminished intensity in the 90 keV to 130 keV region.

TABLE 1
Quantitative Values for the Spectral Changes of
Tungsten Filters as shown in FIGS. 3 and 4A-4C.

| Tungsten Filter | 0 mm | 0.5 mm | 0.7 mm | 1 mm |
|---|---|---|---|---|
| 90-130 keV | 14% | 12% | 8.4% | 4.9% |
| 130-200 keV | 6% | 55.9% | 69.9% | 83% |

A second action in shaping the beam profile can include using a high enough operating voltage on the x-ray tube to acquire the needed intensity of x-rays in the region above 130 keV. The higher the voltage of the x-ray tube, the lower will be the needed beam power to obtain the same sensitivity, and the smaller will be the skin entrance dose.

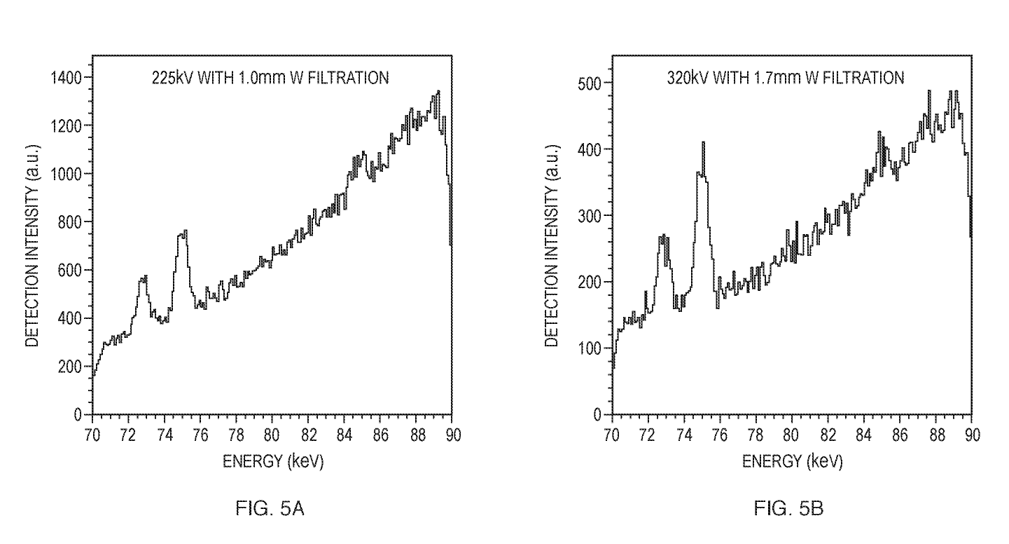

FIGS. 5A-5B show the 70 keV to 90 keV portion of a simulated, detected fluorescence energy spectrum of a bone containing 225 ppm of Pb. The W-anode x-ray tube is operating at 225 kV with 1 mm of tungsten filtration (FIG. 5A) and at 320 keV with 1.7 mm tungsten filtration (FIG. 5B). Both operate at the same power, so the tube current at 320 keV is only 70% of that at 225 keV. Nevertheless, the signal to noise (S/N) values of the K_{α2} and K_{α1} peaks are improved by more than a factor of 2 by going to the higher voltage.

In practice, the choice of high voltage will be dictated by the holistic design. In particular, the desired sensitivity of the XRF-XPbB instrument will be an important consideration. For example, a 160 keV x-ray tube, with its limited flux in the 130 keV-160 keV region, may be useful for a survey to find levels of lead poisoning above 15 ppm. For evaluations that are sensitive at the 5 ppm level, however, the minimum x-ray energy output from a tube is probably 180 keV, and high voltages that result in photon energies well above 200 keV are preferred.

### Reducing the Radiological Burden by Scanning an Area of the Tibia.

The tibia is one of the longest bones in the body and typically has one of the thinnest of overlaying tissue. Even school age children have tibias that present at least 20 cm² of area with an overlaying thickness sufficiently uniform to be useful. Herein are disclosed two distinct overall embodiments to decrease the radiation burden to any given tissue region by increasing the mass being inspected by XRF-XPbB.

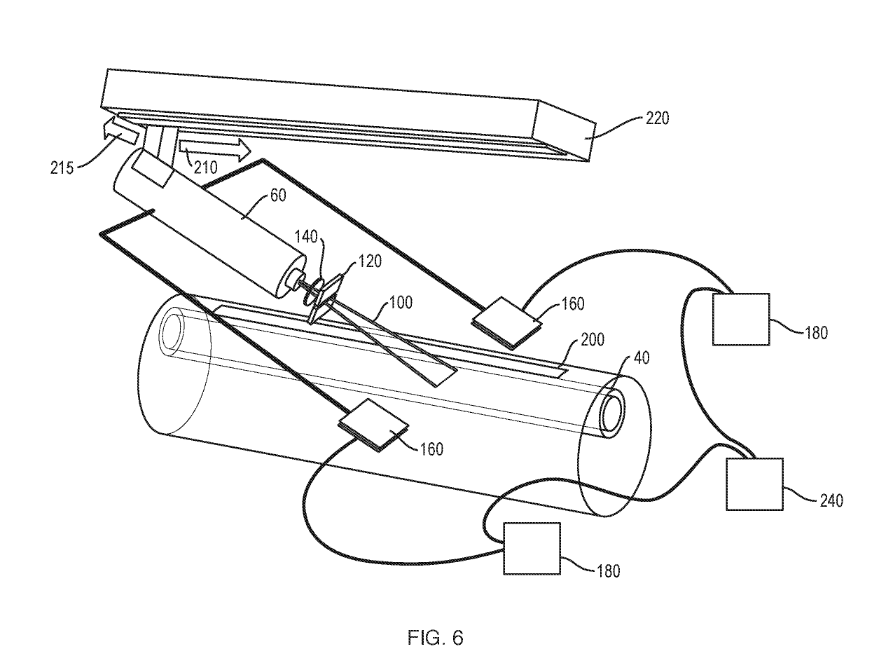

FIG. 6 illustrates main components of one disclosed system. An x-ray source 60 can include an x-ray tube or a mono-block (x-ray tube and power supply packaged together into one assembly). Energy-resolving detectors 160 are collimated so that they only receive radiation emanating directly from the path of the beam in the patient and are mechanically connected to the source. The source/detector assembly can be scanned relative to the patient's tibia 40 by means of an actuator 220 so that the dose to any part of the body is minimized. The source/detector assembly can be scanned along the tibia in the direction indicated by arrow 210, and in addition, the assembly may be scanned across the tibia in the direction indicated by arrow 215. A fan beam from the source is shaped by a filter 140 and formed into the fan beam by a collimator 120. The output of each detector is processed by a dedicated electrical circuit 180, and all the spectral data are processed by processor 240 (also referred to as an analyzer herein), which determines elemental concentrations in the tibia, which can include Pb.

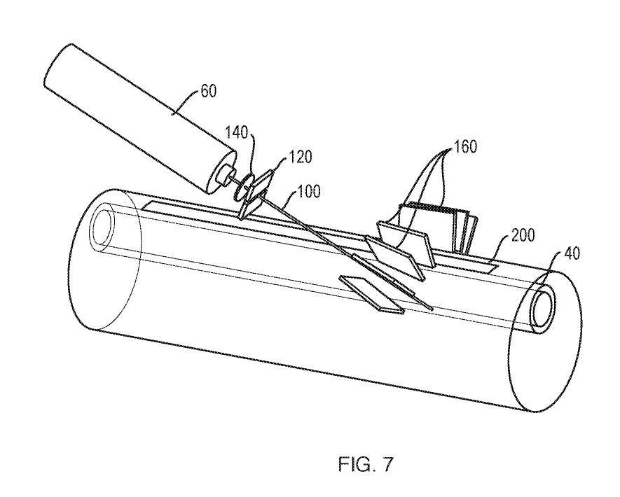

In a first embodiment, referring to FIG. 7, the radiation from the x-ray tube 60 is spectrally shaped by a filter 140 and formed into a pencil beam 100 by collimator 120. The x-ray source, together with the detector assembly, can be moved relative to the tibia 40, such that the area 200 of the patient is raster-scanned by the beam in two dimensions. The detector assembly includes an array of collimated detectors 160 with high energy resolution, that are collimated such that each detector only sees direct scatter or fluorescence emissions that are emitted from the path of the direct beam. One such collimation setup includes strip detectors that are collimated with tungsten plates. With this geometry, many detectors can be placed around the patient's tibia, as shown in FIG. 7. In the simulations that were performed, nine germanium strip detectors were used, but in a clinical system, 20 such detectors can also be used. Note that the scan can be vertical, with the patient seated, or horizontal, with the patient lying down.

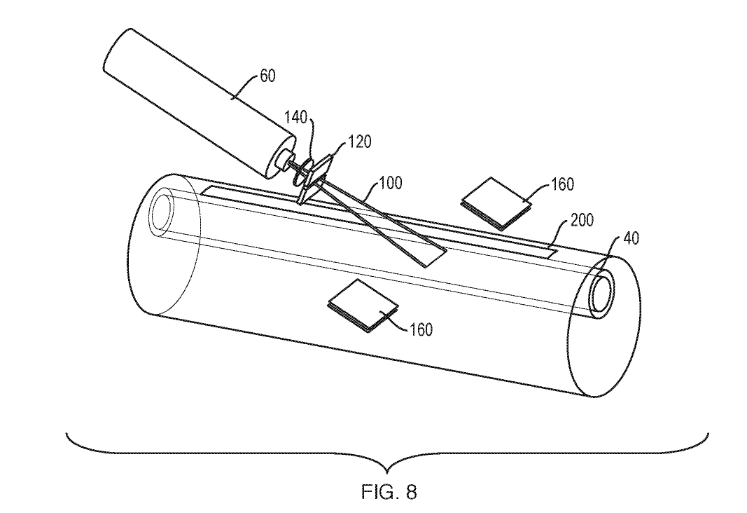

In a second embodiment, referring to FIG. 8, the x-ray beam from the x-ray tube 60 is first formed by collimator 120 into a larger area fan-beam 100 that impinges over a commensurate area 200 of the tibia 40. A pair of collimated detectors 160 are positioned such that the plane of the illuminating fan beam in the tibia lies in the field-of-view of the collimated pair of detectors. The detectors are configured to have good efficiency from all the fluoresced x-rays.

In the embodiment shown in FIG. 7, the Bremsstrahlung beam scans the area in a raster-pattern with a rigid unit including the filtered fluorescing x-ray tube together with a collimated detector array that detects the fluoresced radiation. The x-ray beam has been collimated into a pencil beam that is approximately 2.5 mm by 2.5 mm at the tibia. This severe collimation is made possible by the initial large flux available from the tube. The detector module, which surrounds the patient's tibia, consists of a state-of-the-art array of small detectors, such as CdTe or germanium, each with its own signal processor. The system of source and detectors, as a single unit, scans a 2.5 mm wide by 20 cm long area at a rate of 4 mm/sec, taking 50 seconds. An additional three scans are then taken, with the scanned areas lying next to each other, such that the total area of the patient's tibia that is scanned is 10 mm wide by 20 cm long and with a total measurement time of 200 seconds. An array of 20 CdTe detectors, each with nearly 100% efficiency for 75 keV for fluorescent radiation, and each processing about 5*10⁴ counts/sec, gathers more than 10⁸ counts in a scan. The data may be subdivided into smaller areas to obtain, at less sensitivity, the uniformity of the Pb burden.

In the embodiment shown in FIG. 8, the x-ray beam has been collimated into a fan beam that is approximately 2.5 mm high by 10 mm wide at the tibia. As used herein, the word “collimated” can refer to be partially collimated x-ray beam. For example, in the pencil beam embodiment illustrated in FIG. 7, the pencil beam may still have some divergence, yet may be severely restricted with respect to the x-ray beam emanating from the x-ray tube 60. In this case, the collimated pencil beam 100 may be partially, or nearly completely collimated in both cross-sectional dimensions of the beam, with either minimized divergence or more divergence, yet with less divergence than the beam emanating from the x-ray tube 60. Furthermore, in the embodiment shown in FIG. 8 with the fan beam 100, collimation may be greater, and divergence smaller, in one dimension than in the other cross-sectional dimension, as illustrated in FIG. 8. Thus, in one dimension, divergence and degree of collimation in the fan beam of FIG. 8 may be similar to the divergence and collimation, respectively, in FIG. 7. However, in a perpendicular cross-sectional direction of the fan beam, divergence may be much greater than in the case of the pencil beam of FIG. 7, with correspondingly lesser collimation. In both the embodiments of FIGS. 7 and 8, the beam is collimated, at least in some degree, with respect to the beam from the x-ray tube. It should also be noted that, in some embodiments, substantial collimation can be provided by a collimator built into the x-ray tube, such that a collimator is within the x-ray tube or part of the x-ray tube, and a beam emanating from the x-ray tube, whether a pencil beam, fan beam, or another shaped beam, may be collimated without the external collimator 120.

The detector module consists of a pair of collimated detectors, such as CdTe or germanium, each with its own signal processor. The example system of source and detectors, as a single unit, scans a 10 mm wide by 20 cm long area at a rate of 1 mm/sec, with a total measurement time of 200 seconds.

In one preferred embodiment, a system consistent with has the following specifications:
1. A 500 Watt Bremsstrahlung x-ray source, with a tungsten transmission anode, operating at 225 kV operating voltage.
2. A beam filter, made of material with a K electron binding energy in the range of 60 keV to 70 keV, intercepts the beam to preferentially absorb out the 90 to 130 keV Bremsstrahlung radiation.
3. A collimator of the Bremsstrahlung radiation that produces a shaped beam at the tibia.
4. The Bremsstrahlung beam enters the tibia at an acute angle of less than 35°.
5. A detector array of efficient, high resolution, high count rate detectors, such as CdTe, each with its own signal processor.
6. The detector elements of the array are highly collimated so that they can only detect single Compton scatter events or fluorescent radiation emanating from the beam path in the tibia.
7. The detector array preferentially accepts only the x-rays that have fluoresced in the forward direction.
8. The detector array has a solid angle acceptance of at least a steradian.
9. The multiple array strips and x-ray tube are fixed together as a single inspection unit.
10. The single unit scans a 1 cm wide by 20 cm long area of the tibia in approximately 200 seconds, dwelling approximately 6 seconds and 25 seconds on a given point for the embodiments of FIGS. 7 and 8, respectively.

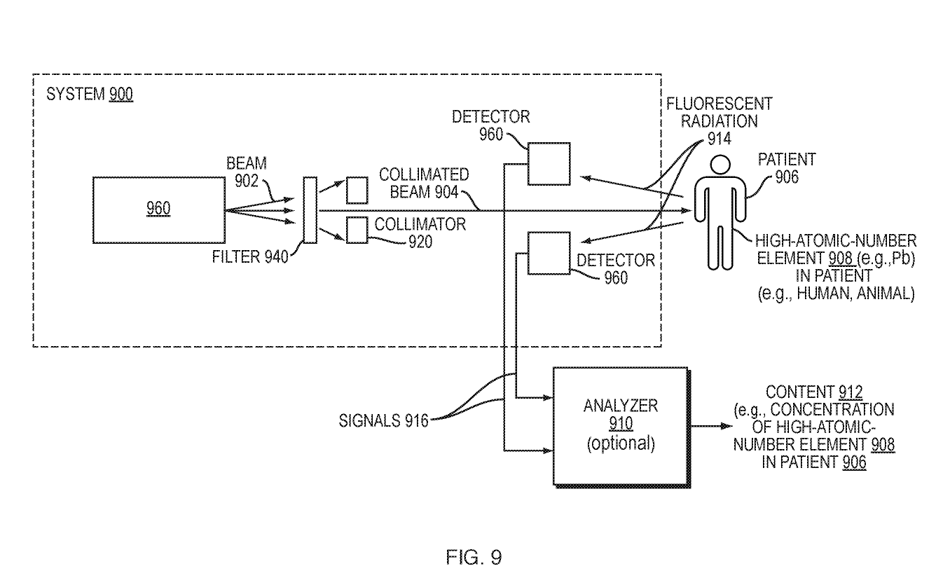

FIG. 9 is a schematic diagram illustrating a system 900 for detecting one or more high-atomic-number elements 908 in a patient 906. The high-atomic-number element 908 can include lead, for example, and the patient 906 can be a human, animal, etc. Furthermore, the element 908 can be in a portion of the patient 906, such as a tibia bone, soft tissue, another bone, or another portion of the body. There are some advantages of detecting the element 908 in the tibia bone of a human, as described herein above.

The system 900 includes a Bremsstrahlung x-ray source 960. A typical example Bremsstrahlung x-ray source includes an x-ray tube. An example of a Bremsstrahlung radiation energy spectrum is provided in FIG. 3, for example. However, another source that is capable of producing x-rays by the Bremsstrahlung effect can be used. The x-ray source 960 is configured to produce the x-rays in a beam 902, with the x-rays being in an energy spectrum including an energy of at least 160 keV. As is known, an x-ray tube, for example, outputs broadband x-rays, in contrast to a radioactive isotope, which typically outputs x-rays or gamma rays with more well-defined energies.

The system 900 also includes a filter 940 that is configured to absorb x-rays from the source 960 in a particular region of the energy spectrum output from the source. This particular region over which the filter 940 absorbs x-rays can be in an energy range of approximately 88-130 keV, for example, as described hereinabove. Examples of filtered, attenuated Bremsstrahlung radiation energy spectra are shown in FIGS. 4A-4C.

In some embodiments, this absorption range of the filter corresponds to an energy region where there is an increased cross section for producing x-ray fluorescence. The filter 940 may include any of the material thicknesses, compositions, element atomic numbers, and other specifications described herein for various embodiments.

The system 900 also includes a collimator 920 that is configured to constrict divergence of the x-ray beam 902 to produce a beam 904 that is, at least in part, collimated. As described hereinabove, a collimated beam may be partially collimated, while having greater divergence in another cross-sectional dimension, such as is in the case of a fan beam. In other embodiments, the collimated beam 904 may be a pencil beam and may be either highly collimated into dimensions or partially collimated in two dimensions.

The system 900 also includes one or more detectors 960. These detectors can include detector materials and configurations as described herein in connection with any embodiment. The one or more detectors 960 are collimated, energy-resolving x-ray detectors that are configured to detect fluorescent radiation 914 emitted from one or more high-atomic-number elements 908 in the patient.

The fluorescent radiation 914 is emitted from the element 908 in response to the collimated beam 904 that is incident on the patient 906. In one example, the fluorescent radiation 914 can include K alpha 1, K alpha 2, and K beta 1,3 x-ray fluorescence radiation from lead, as illustrated in the spectrum shown in FIG. 2. Such K-shell excitations occur in lead when an x-ray beam, such as the collimated beam 904, is incident on a patient. The x-ray beam can penetrate soft tissue and particularly enter a bone of the patient, such as a tibia bone, and cause lead or another high-atomic-number element to fluoresce.

Also illustrated in FIG. 9 is an analyzer 910 (optional) that is configured to receive signals from the detectors 960, such as electronic signals, that are representative of the fluorescent radiation 914 emitted from the patient and detected by the detectors 960. The analyzer 910 is configured to process the signals 916 to determine a content of the one or more elements 908 in the patient. Such content 912 can be a concentration, such as a concentration as low as 5 ppm. However, contents 912 may be provided in other forms, such as volumetric or number density contents, for example. In some embodiments, the system 900 includes the optional analyzer 910. It should also be noted that, as described herein above, in some embodiments, one or both of the filter 940 and collimator 920 can be incorporated into the x-ray source 960, such that the x-ray source, filter, and a collimator are part of a single module.

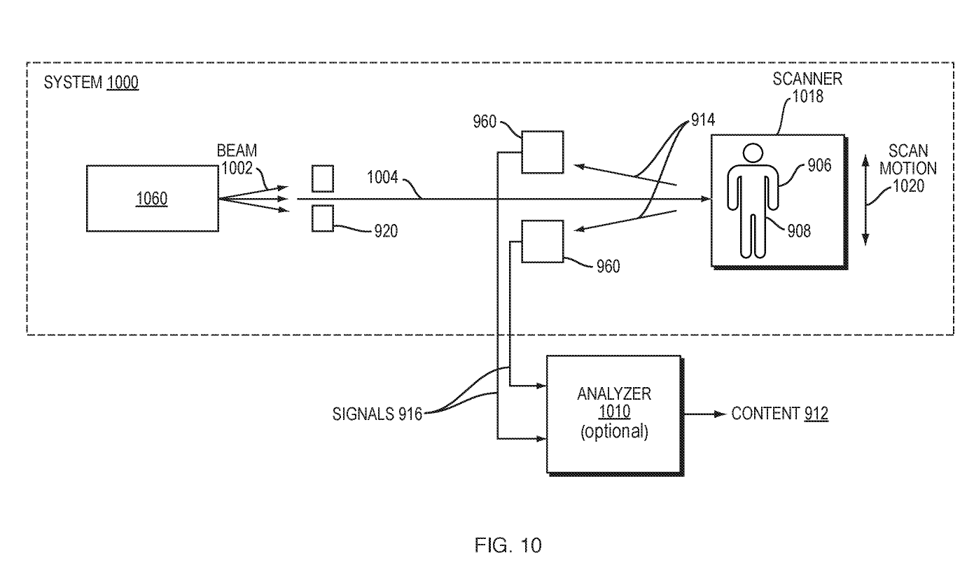

Furthermore, as described hereinabove, and as described in connection particularly with FIGS. 6 and 10, many embodiments include a scanner that is configured to cause relative motion between the patient 906 and the x-ray beam 904 incident on the patient in order to scan at least a portion of the patient with the x-ray beam 904. The portion of the patient can include a tibia bone, for example, or another portion of the body. A scanner may be a patient table that is configured to translate a patient, or a part of the patient, with respect to a stationary collimated beam 904. However, in other embodiments, such as that illustrated in FIG. 6, the scanner may be an actuator configured to translate the beam 904 with respect to the patient, along either one dimension (e.g., such as in the case of a fan beam of the size of the tibia bone width configured to be scanned along the length of the tibia bone) or in two dimensions, such as in the case of raster scanning a pencil beam to intersect with various parts of the patient or part of the patient such as the tibia bone.

FIG. 10 is a schematic diagram illustrating a system 1000 for detecting one or more high-atomic-number elements in a patient. The system 1000 differs from the system 900 illustrated in FIG. 9, in that an x-ray source 1060 that is configured to produce x-rays can be a narrowband x-ray source, such as a radioactive isotope sample, for example. Certain radioactive isotopes are described herein above, for example. However, the sample can include any isotope that is capable of excitation of the high-atomic-number element 908 in the patient to emit the fluorescent radiation 914. Such excitation can include K-shell excitation in the element 908. The collimator 920 collimates a beam 1002 from the source 1060, which can include narrowband x-ray radiation, such as that output from a radioactive isotope. The collimator 920 receives the beam 1002 and outputs a collimated beam 1004 to be incident on the patient 906. As in the embodiment of FIG. 9, fluorescent radiation 914, which is emitted from the element 908 in the patient in response to the collimated beam 1004 incident on the patient, is detected by one or more detectors 960. Signals 916 output from the detectors 960 and received by an optional analyzer 1010. The analyzer 1010, which can be part of an embodiment system in certain embodiments, functions as the analyzer 910 illustrated in FIG. 9. In particular, the analyzer 1010 is configured to output a content 912, which can include a concentration of the element 908 in the patient.

The system 1000 also includes a scanner 1018 that is configured to translate the patient 906, or a portion of the patient, such as a leg, with respect to the collimated beam 1004, with example scan motion 1020, in order to scan at least a portion of the patient 906 with the collimated x-ray beam 1004. The scan motion 1020 may be in one dimension or two dimensions, for example. Furthermore, the scan motion may be in three dimensions in certain embodiments.

In some embodiments, the scanner 1018 is a patient table on which the patient lays, which translates the patient with respect to the beam. However, many various actuators are known and can be configured to hold a leg of the patient 906 and translate only the leg, for example. Furthermore, it embodiments such as that shown in FIG. 6, the scanner 1018 is not configured to translate the patient, but is instead configured to translate the beam 1004 with respect to the patient to provide the relative motion in one or two dimensions. Such a scanner may include a scanner configured to move the entire system including the x-ray source 1060, collimator 920, and detectors 960 with respect to the patient. Furthermore, in some embodiments, the scanner scans the collimator 920 with respect to both the x-ray source 1060 and patient 908 to cause the relative motion for scanning, effectively blocking parts of the beam 1002, selectively, at different times, in order to provide the scan.

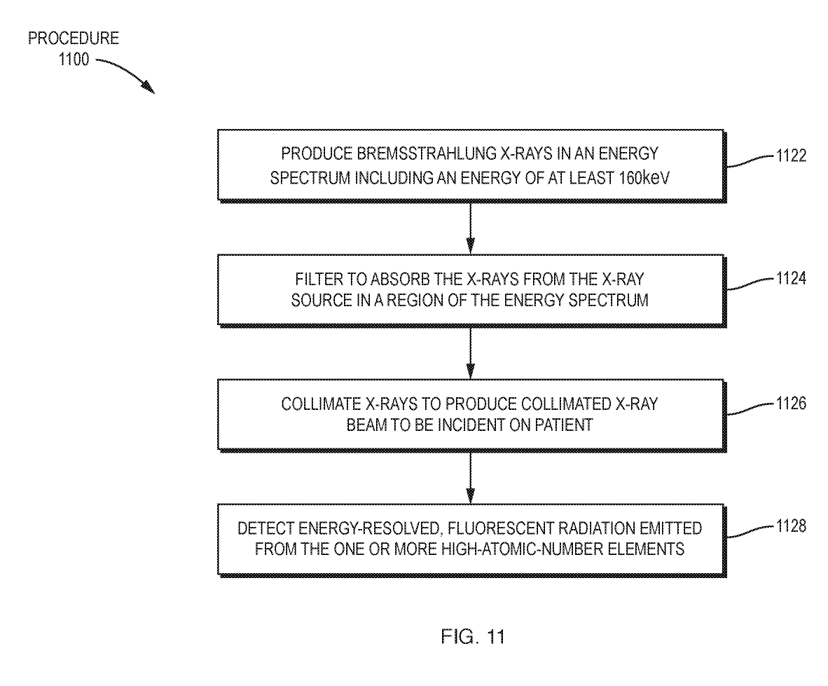

FIG. 11 is a procedure 1100 illustrating a method for detecting one or more high-atomic-number elements in a patient. At 1122, Bremsstrahlung x-rays are produced in an energy spectrum including an energy of at least 160 keV. At 1124, filtering is performed on the x-rays to absorb the x-rays from the x-ray source in a region of the energy spectrum.

At 1126, the x-rays from the x-ray source are collimated to produce a collimated x-ray beam to be incident on a patient. At 1128, energy-resolved, fluorescent radiation emitted from the one or more high-atomic-number elements in the patient in response to the collimated x-ray beam incident on the patient is detected.

The procedure 1100 may be performed, for example, by the system 900 illustrated in FIG. 9, or by any one of the systems illustrated in FIGS. 6-8, for example. Furthermore, in other embodiments similar to the procedure 1100, procedures may include any of the optional elements or actions described herein with respect to various embodiments. For example, other procedures can include scanning by producing relative motion between the patient and the x-ray beam to scan at least a portion of the patient with the x-ray beam.

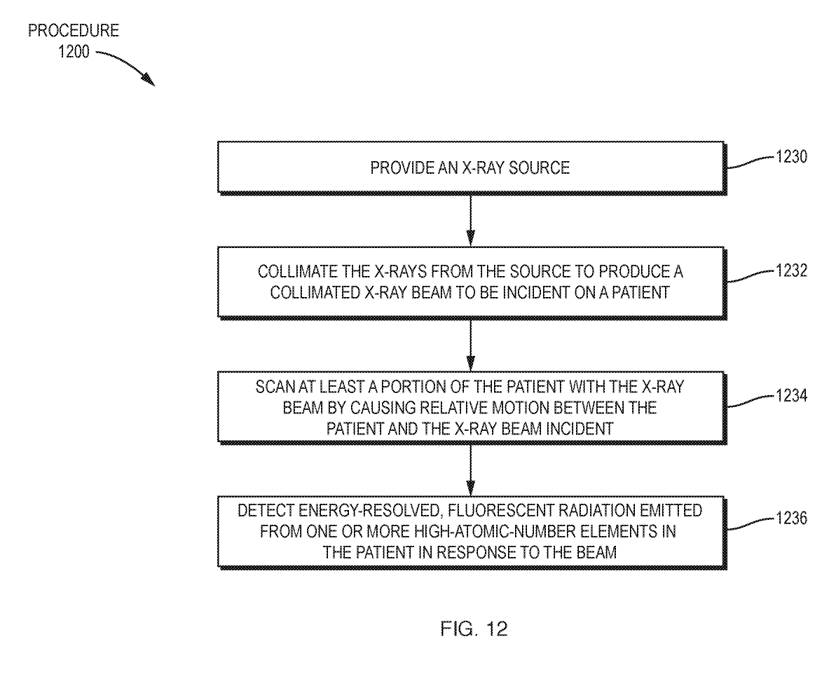

FIG. 12 is a flow diagram illustrating a procedure 1200 for detecting one or more high-atomic-number elements in a patient. At 1230, an x-ray source is provided. The x-ray source can include a broadband source, such as an x-ray tube or other Bremsstrahlung x-ray radiation source, or a narrowband x-ray source, such as a radioactive isotope.

At 1232, the x-rays from the x-ray source are collimated to produce a collimated x-ray beam to be incident on a patient. At 1234, at least a portion of the patient is scanned with the x-ray beam by causing relative motion between the patient and the x-ray beam incident on the patient. At 1236, energy resolved, fluorescent radiation is detected, where the fluorescent radiation is emitted from one or more high-atomic-number elements in the patient in response to the collimated x-ray beam incident on the patient.

In other embodiments that are similar to procedure 1200, other procedural elements may also be performed, consistent with embodiments described in the specification. In one example, a procedure similar to the procedure 1200 may also include filtering the x-ray beam from the x-ray source to attenuate at least a portion of a spectrum of x-ray energies provided by the x-ray source. Such attenuation of a portion of the spectrum is particularly helpful where the x-ray source is a broadband x-ray source, such as an x-ray tube or other Bremsstrahlung radiation source, and such attenuation can

Items within the scope of claimed and described embodiments:
1. A system designed to take an in-vivo measurement of the content of high-atomic-number elements in a patient, the system comprising: a) an x-ray tube with an operating voltage of at least 160 kV; b) a collimator to allow a beam of radiation to be incident on the patient; c) an array of one or more collimated energy resolving detectors to detect fluorescent radiation from high-atomic-number elements contained within the patient's body.
2. A system according to item 1, wherein the element being measured is lead.
3. A system according to item 1, wherein the x-ray beam is shaped with a filter consisting of a material with an atomic number of at least 50.
4. A system according to item 3, wherein the filter consists of a material with an atomic number in the range of 72-92.
5. A system according to item 4, wherein the filter is at least 0.5 mm thick.
6. A system according to item 1, wherein the collimated x-ray beam is a pencil beam
7. A system according to item 1, wherein the collimated x-ray beam is a fan beam
8. A system according to item 1, wherein the x-ray beam is raster-scanned over a two-dimensional area of the patient's body
9. A system according to item 1, wherein the x-ray beam is scanned along one dimension of the patient's body
10. A system according to item 6, wherein multiple collimated detectors are arranged to only detect radiation emanating from the path of the pencil beam in the patient's body
11. A system according to item 7, wherein multiple collimated detectors are arranged to only detect radiation emanating from the path of the fan beam in the patient's body

The following four references are hereby incorporated herein by reference in their entireties:
In vivo X-ray fluorescence of lead in bone: review and current issues. A. C. Todd and D. R. Chettle. Environmental Health Perspectives, 1994 February, 102(2):172-177.
Studies in Bone Lead: A New 109Cd K-XRF Measuring System. Huiling Nie, PhD Thesis, McMaster University. 2005.
Application and Methodology of in-vivo K x-ray Fluoresence of Pb in Bone. Huiling Nei, Howard Hu and David R. Chettle. X-Ray Spectrometry Vol. 37, January/February 2008
Bone Lead Measured by x-ray Fluorescence: Epidemiologic Methods. Howard Hu, Antonio Aro and Andrea Rotnitzky”)

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A system for detecting one or more high-atomic-number elements in a patient, the system comprising:
a Bremsstrahlung x-ray source configured to produce x-rays in an energy spectrum including an energy of at least 160 keV;
a filter configured to absorb the x-rays from the x-ray source in a region of the energy spectrum;
a collimator configured to receive the x-rays from the x-ray source and to output a collimated x-ray beam to be incident on a patient; and
one or more collimated, energy-resolving x-ray detectors configured to detect fluorescent radiation emitted from one or more high-atomic-number elements in the patient in response to the collimated x-ray beam incident on the patient.

2. The system of claim 1, further comprising a scanner configured to cause relative motion between the patient and the x-ray beam incident on the patient in order to scan at least a portion of the patient with the x-ray beam.

3. The system of claim 2, wherein the scanner is configured to move the patient with respect to the x-ray beam to cause the relative motion.

4. The system of claim 2, wherein the scanner is further configured to cause relative one-dimensional motion between the patient and the x-ray beam to scan the portion of the patient along one dimension.

5. The system of claim 2, wherein the scanner is further configured to cause relative two-dimensional motion between the patient and the x-ray beam to scan the portion of the patient along two dimensions.

6. The system of claim 1, further comprising an analyzer configured to receive signals from the one or more detectors, the signals representative of the fluorescent radiation emitted and detected, wherein the analyzer is further configured to process the signals to determine a content of the one or more high-atomic-number elements in the patient.

7. The system of claim 6, wherein the analyzer is further configured to determine the content of the one or more high-atomic-number elements with concentration as low as 5 parts per million (ppm).

8. The system of claim 1, wherein the filter is further configured to absorb the x-rays from the x-ray source in a region of the energy spectrum corresponding to x-rays Compton scattered from the patient in response to the collimated x-ray beam incident on the patient, such that a signal-to-background ratio can be enhanced.

9. The system of claim 1, wherein the filter comprises a material with an atomic number of at least 50.

10. The system of claim 9, wherein the filter further comprises a material with an atomic number in a range of about 72-92.

11. The system of claim 1, wherein the filter has a thickness of at least 0.5 mm.

12. The system of claim 1, wherein the one or more high-atomic-number elements include lead.

13. The system of claim 1, wherein the collimated x-ray beam is a pencil beam.

14. The system of claim 13, wherein the one or more collimated detectors are arranged to detect the fluorescent radiation emitted only from a path of the pencil beam in the patient.

15. The system of claim 1, wherein the collimated x-ray beam is a fan beam.

16. The system of claim 15, wherein the one or more collimated detectors are arranged to detect the fluorescent radiation emitted only from a path of the fan beam in the patient.

17. A method for detecting one or more high-atomic-number elements in a patient, the method comprising:
producing Bremsstrahlung x-rays in an energy spectrum including an energy of at least 160 keV;
filtering to absorb the x-rays from the x-ray source in a region of the energy spectrum;
collimating the x-rays from the x-ray source to produce a collimated x-ray beam to be incident on a patient; and
detecting energy-resolved, fluorescent radiation emitted from one or more high-atomic-number elements in the patient in response to the collimated x-ray beam incident on the patient.

18.-33. (canceled)

34. A system for detecting one or more high-atomic-number elements in a patient, the system comprising:
an x-ray source configured to produce x-rays;
a collimator configured to receive the x-rays from the x-ray source and to output a collimated x-ray beam to be incident on a patient;
a scanner configured to cause relative motion between the patient and the x-ray beam incident on the patient in order to scan at least a portion of the patient with the x-ray beam; and
one or more collimated, energy-resolving x-ray detectors configured to detect fluorescent radiation emitted from one or more high-atomic-number elements in the patient in response to the collimated x-ray beam incident on the patient.

35. The system of claim 34, wherein the scanner is configured to move the patient with respect to the x-ray beam to cause the relative motion.

36. The system of claim 34, wherein the scanner is configured to translate the x-ray beam with respect to the patient.

37. The system of claim 34, wherein the scanner is further configured to cause relative one-dimensional motion between the patient and the x-ray beam to scan the portion of the patient along one dimension.

38. The system of claim 34, wherein the scanner is further configured to cause relative two-dimensional motion between the patient and the x-ray beam to scan the portion of the patient along two dimensions.

39. The system of claim 34, wherein the x-ray source is a radioactive isotope.

40. The system of claim 34, wherein the x-ray source is an x-ray tube.

41. The system of claim 34, further comprising an analyzer configured to receive signals from the one or more detectors, the signals representative of the fluorescent radiation emitted and detected, wherein the analyzer is further configured to process the signals to determine a content of the one or more high-atomic-number elements in the patient.

42. The system of claim 41, wherein the analyzer is further configured to determine the content of the one or more high-atomic-number elements with concentration as low as 5 parts per million (ppm).

43. The system of claim 34, wherein the one or more high-atomic-number elements include lead.

44.-54. (canceled)
